(19) United States
(12) Patent Application Publication
(10) Pub. No.: US 2017/0020446 A1
(43) Pub. Date: Jan. 26, 2017

(54) SYSTEMS, METHODS AND APPARATUSES FOR MONITORING HYPOXIA EVENTS

(71) Applicant: HALARE, INC., WARRIORS MARK, PA (US)

(72) Inventors: ANTHONY C. WARREN, WARRIORS MARK, PA (US); KYLE H. GOLDSCHMIDT, SAINT LOUIS PARK, MN (US)

(21) Appl. No.: 15/303,126

(22) PCT Filed: Apr. 10, 2015
(86) PCT No.: PCT/US2015/025231

§ 371 Date: Oct. 10, 2016

Related U.S. Application Data
(60) Provisional application No. 61/978,458, filed on Apr. 11, 2014.
(60) Provisional application No. 62/038,777, filed on Aug. 18, 2014.

(51) Int. Cl. A61B 5/00; A61B 5/11; A61B 7/00; A61B 5/145; A61B 5/0205

(52) U.S. Cl. CPC A61B 5/4818; A61B 5/14542; A61B 5/0205; A61B 7/003; A61B 5/11; A61B 5/7282; A61B 5/7257; A61B 5/7275; A61B 5/02416

(57) ABSTRACT

Systems and methods for monitoring hypoxia in a user over a period of time are disclosed. A sensor assembly detects data indicative of at least one physiological parameter of the user and an electronic computing unit analyzes the data indicative of the at least one physiological parameter of the user detected by the sensor assembly and determines, from the analyzed data, an occurrence of a hypoxia event. The electronic computing unit may additionally determine characteristics of the data indicative of the at least one physiological parameter associated with the determined occurrence of the hypoxia event and determine trends from changes in the determined characteristics.

### CROSS-REFERENCE TO RELATED APPLICATIONS

This application claims priority to U.S. Provisional Application No. 61/978,458 entitled “Systems, Methods, and Apparatus for Monitoring Periodic Mild Hypoxia” filed on Apr. 11, 2014, and U.S. Provisional Application No. 62/038,777 entitled “Methods and Apparatuses for Monitoring Hypoxia Events” filed on Aug. 18, 2014, the contents of which are incorporated fully herein by reference.

### FIELD OF THE INVENTION

The present invention relates to breath training and detection and monitoring of hypoxia events.

### BACKGROUND OF THE INVENTION

A significant portion of sleep disordered breathing is a condition characterized by repeated episodes during sleep resulting in many detrimental and detectable effects on a person. Research has shown that sleep disordered breathing can have major short term and long term deleterious impacts. Therefore, there exists a need for improved and accessible systems and methods for detecting and monitoring sleep disordered breathing, such as hypoxia events, in persons and providing instructions or regimens to improve sleep disordered breathing in persons, as well as determining the effectiveness of such instructions or regimens.

### SUMMARY OF THE INVENTION

Aspects of the invention are embodied is systems and methods for monitoring hypoxia in a user over a period of time. A sensor assembly may detect data indicative of at least one physiological parameter of the user and an electronic computing unit may analyze the data indicative of the at least one physiological parameter of the user detected by the sensor assembly and determine, from the analyzed data, an occurrence of a hypoxia event. The electronic computing unit may additionally determine characteristics of the data indicative of the at least one physiological parameter associated with the determined occurrence of the hypoxia event and determine trends from changes in the determined characteristics.

### BRIEF DESCRIPTION OF THE DRAWINGS

The invention is best understood from the following detailed description when read in connection with the accompanying drawings, with like elements having the same reference numerals. When a plurality of similar elements is present, a single reference numeral may be assigned to the plurality of similar elements with a capital letter designation referring to specific elements. Included in the drawings are the following figures:

FIG. 1 is a block diagram of a system for monitoring hypoxia events in persons according to aspects of the invention;

FIGS. 2A and 2B are block diagram depicting sensor assemblies in accordance with aspects of the invention;

FIG. 3 is a graph showing a desaturation period in accordance with aspects of the invention;

FIG. 4 is a graph depicting detection of breath holding according to aspects of the invention;

FIG. 5 is a plot showing desaturation events in a user in accordance with aspects of the invention;

FIGS. 6A and 6B depict data regarding PPG amplitudes according to aspects of the invention;

FIG. 7 is a graph showing normalized values of blood oxygen saturation and heart rate in accordance with aspects of the invention;

FIG. 8 shows HRV with good coherence according to aspects of the invention;

FIG. 9 shows HRV with poor coherence in accordance with aspects of the invention;

FIG. 10 is a graph showing calculation of a Q factor according to aspects of the invention;

FIG. 11 is a graph showing a trend in breath hold time in accordance with aspects of the invention;

FIG. 12 is a graph showing a trend in Q factor in accordance with aspects of the invention;

FIG. 13 is a graph showing a trend in breaths per minute according to aspects of the invention;

FIG. 14 is a graph showing a trend in change in heart rate in accordance with aspects of the invention;

FIG. 15 is a graph depicting correlation to show hypoxic events in accordance with aspects of the invention;

FIG. 16 is a graph depicting trends in IBI and blood oxygen saturation in accordance with aspects of the invention; and

FIG. 17 is a flowchart of steps in a method for monitoring hypoxia in accordance with aspects of the invention.

### DETAILED DESCRIPTION OF THE INVENTION

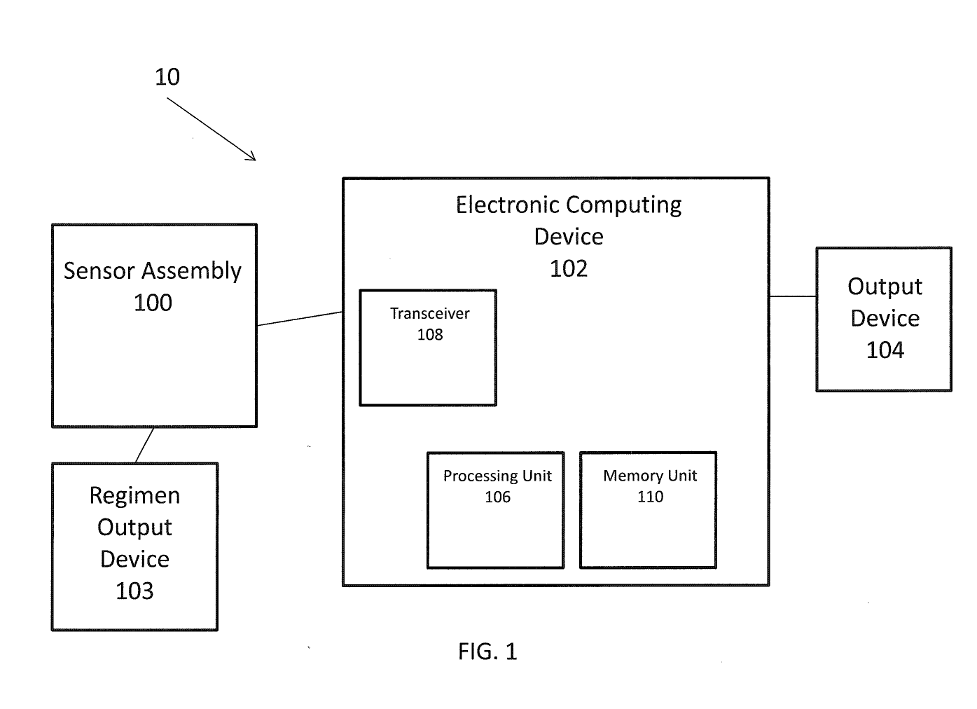

Referring to FIG. 1, a block diagram of a system 10 for monitoring hypoxia events (e.g., during sleep and/or breath training) and for determining effectiveness of breath training regimens on hypoxia events according to aspects of the invention. Other aspects of the invention include monitoring and/or determining one or more other parameters such as whether an individual is snoring or in a particular position (e.g., laying on back).

The system 10 includes a sensor assembly 100, an electronic computing device 102, and an output device 104. Although the sensor assembly 100, the electronic computing device 102, and output device 104 are depicted as separate components in system 10, it is contemplated that any or all of these components may be integrated together in two or one device. For example, the sensor assembly, the electronic computing device 102, and the output device 104 may be integrated into an apparatus attachable to a user (e.g., a wristband, a neckband, other attachments, etc.), or in a smart device, such as a smart phone, tablet computer, laptop computer, etc.

The sensor assembly 100 includes at least one sensor that is configured to detect physiological data of the user that can be used to detect hypoxia events during breathing of the user both during breath training regimens and while the user is asleep. The sensor assembly 100 may include, for example, an accelerometer, a blood oxygen saturation sensor, a motion sensor, an audio sensor, a heart rate sensor, a breath sensor, a position sensor, a photoplethsysmographic signal (PPG) sensor etc. Other suitable sensors for detecting physiological data of a user will be understood by one of ordinary skill in the art from the description herein.

As referred to herein, a “user” is a person or persons or other entity undergoing treatment, e.g., for sleep disordered breathing (SDB), wherein the treatment preferably includes breath training sessions. During breath training sessions, the user may receive instructions.

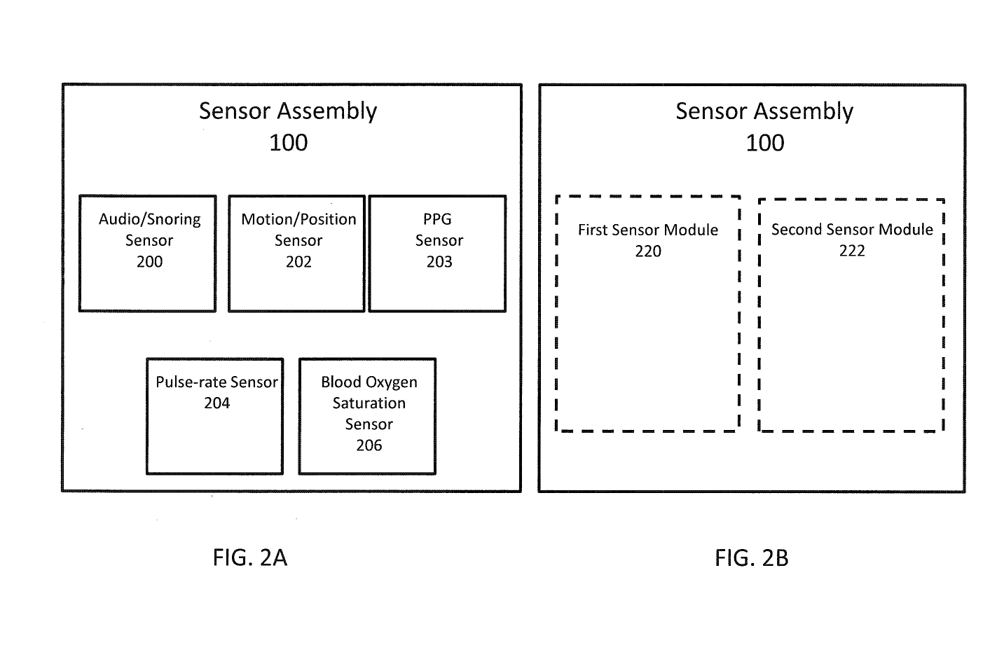

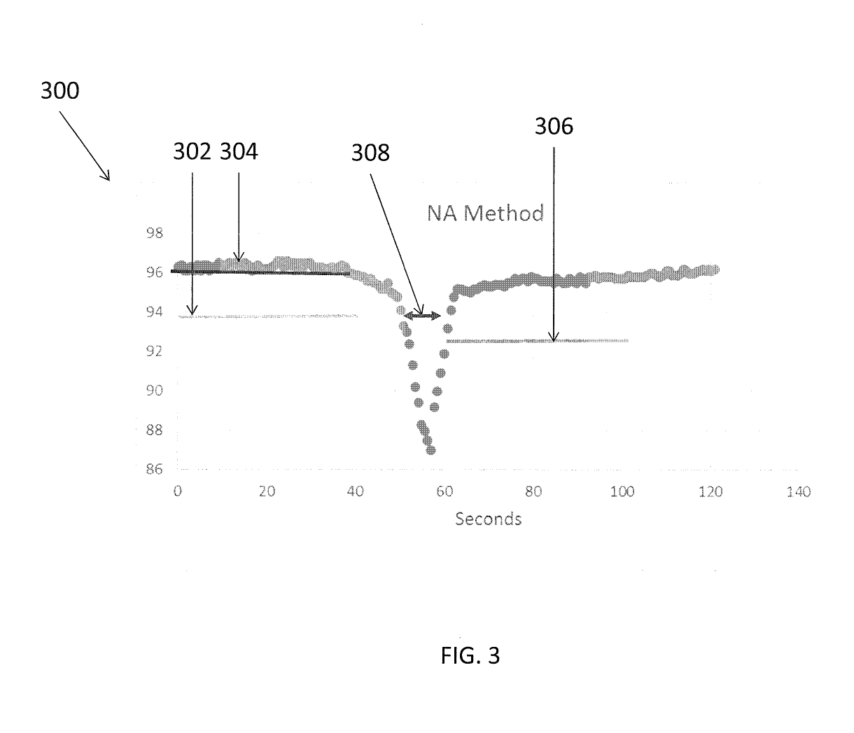

Referring to FIG. 2A, a diagram of an example of a sensor assembly 100 is shown. Although the sensor assembly 100 is shown with multiple sensors, the sensor assembly 100 may include only one of the sensors, or any combination of the sensors shown. In one embodiment, the sensor assembly 100 includes an audio/snoring sensor 200 adapted to detect snoring sounds from the user. The snoring sensor 200 may be adapted to detect time intervals between snoring sounds of the user and to detect the intensity of snoring from the user. Analysis of the physiological data in the form of intervals of snoring sounds and intensity of snoring sounds may be used to differentiate between apneic users suffering from Obstructive Sleep Apnea (OSA) and benign persons. Detection of snoring sounds is useful to determine whether a user has a history of snoring, whether snoring is an indication of possible SDB or is generally benign, whether the snoring is an indication of OSA, where in the user airway constrictions are occurring, etc.

The sensor assembly 100 may also include a position/movement sensor 202. In one embodiment, the position/movement sensor 202 is adapted to detect restlessness in sleep of the user as an indicator of periods of apnea or hypopnea in a user. In one example, OSA symptoms often exacerbate when the user lies on their back. Periods of excessive movement of the user during sleep may also be detected by the position/movement sensor 202, such that timing and intensity of the restlessness of the user may be analyzed to detect sleep disordered events for further analysis.

The sensor assembly 100 may also include a PPG sensor 203. During hypoxic episodes, the amplitude of the PPG signal declines. Thus, the length of time of the hypoxic event as well as the time of occurrence of the hypoxic event can be observed by detecting changes in amplitude of the PPG signal, which may be detected from the PPG sensor 203.

The sensor assembly 100 may also include a pulse-rate sensor 204 for detecting the heart rate of the user during sleep and/or during breathing exercises. The pulse-rate sensor 204 may be configured to detect periods of accelerations and/or decelerations in pulse rate of the user during sleep and/or breath training exercises, as well as disturbed or erratic pulse rates of the user as sleep disordered events, which may be used for further analysis.

A blood oxygen saturation (O2sat) sensor 206 may also be included in the sensor assembly 100. In an embodiment, the O2sat sensor 206 is adapted to detect changes in oxygen concentration levels of the user's blood during breath training exercises while awake, during sleep, or as potential sleep disordered events.

FIG. 2B depicts an example of a sensor assembly 100 that includes a first sensor module 220 and a second sensor module 222. The first sensor module 220 and second sensor module 222 may be integrated into a single sensor apparatus, or may be integrated into separate components. In one embodiment, the first sensor module 220 includes sensors that are adapted to detect physiological data from a user during breath training sessions while the user is awake, and the second sensor module 222 includes sensors that are adapted to detect sleep disordered breathing of the user while the user is asleep, or vice versa. Each of the sensor modules 220 and 222 may include one or any combination of sensors such as those described above.

Referring back to FIG. 1, the system 10 includes a regimen output device 103. The regimen output device 103 is adapted to output instructions to the user according to the breath training regimen established for the user to detect physiological data of the user while the user is awake. The regimen output device 103 may be coupled to the sensor assembly 100, or may be a separate component, such as a smart phone, tablet computer, laptop computer, or other communication device capable of providing breath training instructions to the user.

The system 10 further includes an electronic computing device 102 with a processing unit 106, a transceiver 108, and a memory unit 110. The transceiver 108 may be utilized to receive physiological data detected from the sensor assembly 100. In embodiments where the electronic computing device 102 is integrated with the sensor assembly 100, the transceiver 108 may not be a necessary component for the transmission and reception of data to be analyzed by the electronic computing device 102. The memory unit 110 is depicted as integrated into the electronic computing device 102. It is contemplated that additional memory units may be utilized, such as a memory unit integrated into the sensor assembly 100 or a cloud storage device. Such memory units are configured to store detected physiological data and subsequent analyzed data.

The processing unit 106 is adapted to process the data detected by the sensor assembly 100 according to particular algorithms to detect sleep disordered events, determine whether sleep disordered events are indicative of SDB symptoms, detect physiological data from the user produced in response to a breath training regimen, and to determine the effectiveness of the breath training regimen on the user based on the detection of sleep disordered events.

The particular algorithms the processing unit 106 applies to the physiological data detected by the sensor assembly 100 depend upon the type of data detected and the sensors that are used to detect the data. Although the algorithms described herein are related to an individual sensor type, the physiological data analyzed from each type of sensor may be used in conjunction with or in combination with data from other sensors to detect sleep disordered events, and develop trends of the user over time to determine effectiveness of a breath training regimen on the user.

In examples where the sensor assembly 100 includes an audio/snoring sensor, such as sensor 200, the processing unit 106 may apply algorithms as follows. Baselines may be established for time intervals between snoring of the user and intensity of the snoring for the user. The processing unit 106 receives the snoring physiological data and tracks the snoring of the user. When the snoring sensor 200 records snoring occurrences of the user that occur within a time interval that exceeds the time interval baseline, the processing unit 106 determines that the exceeding of the baseline is a sleep disordered event. Alternatively, the time interval baseline may be established such that the processing unit 106 determines a sleep disordered event if snoring occurrences of the user occur too quickly within one another. Similarly, the audio level of the snoring (e.g., a decibel level) may be indicative of intensity of the snoring of the user. A baseline may be established such that when the audio level of a snoring occurrence of the user exceeds the baseline, the intensity of such snoring occurrence may be determined to be a sleep disordered event by the processing unit 106.

In order to record audible snoring sounds with sufficient fidelity to enable the analysis, the sound sensor 200 may be mounted closer to the face of the user, using, as one example, a microphone near the throat. In this case, the sound sensor 200 may be in a separate sensor module apparatus which communicates either by wire, or preferably wirelessly, to either the wrist mounted module, or directly to an electronic computing apparatus for subsequent analysis as described above. This method increases the fidelity of the audible sound detection by reducing interference say from another snorer nearby, or by a wrist mounted sound detector being physically obscured from the user's head.

In examples where the sensor assembly 100 includes a motion or position sensor 202, baselines may be established against which the physiological data from the motion/position sensor 202 is analyzed with the processing unit 106. Amounts of motion or changes of position of the user during sleep are detected by the motion/position sensor 202. When the amounts of motion or changes of position exceed the established baselines, the processing unit 106 determines that the amount of motion or change of position can be a sleep disorder event. Data from the sensor 202 are analyzed using a rolling average and periods of extensive movement from an established resting baseline and lasting more than 10 seconds indicate periods of restlessness the timing and intensity of which are stored together with the time stamp for subsequent correlation. Similarly, position orientation is also stored with a time stamp for subsequent correlation.

Similarly with a pulse rate sensor 204, the processing unit 106 may be adapted to detect heart rate variability (HRV) in the user during sleep. Analysis techniques, such as converting R-R data sets into frequency domain using Fast Fourier Transform algorithms can derive HRV and the time when the power and frequency spectrum of the HRV change. In addition, longer periods of significantly disturbed or erratic HRV may indicate periods of an intensive series of apnea or hypopnea events. The times, amplitudes, and other associated parameters analyzed from the pulse rate sensor 204 may be used for later correlation analysis.

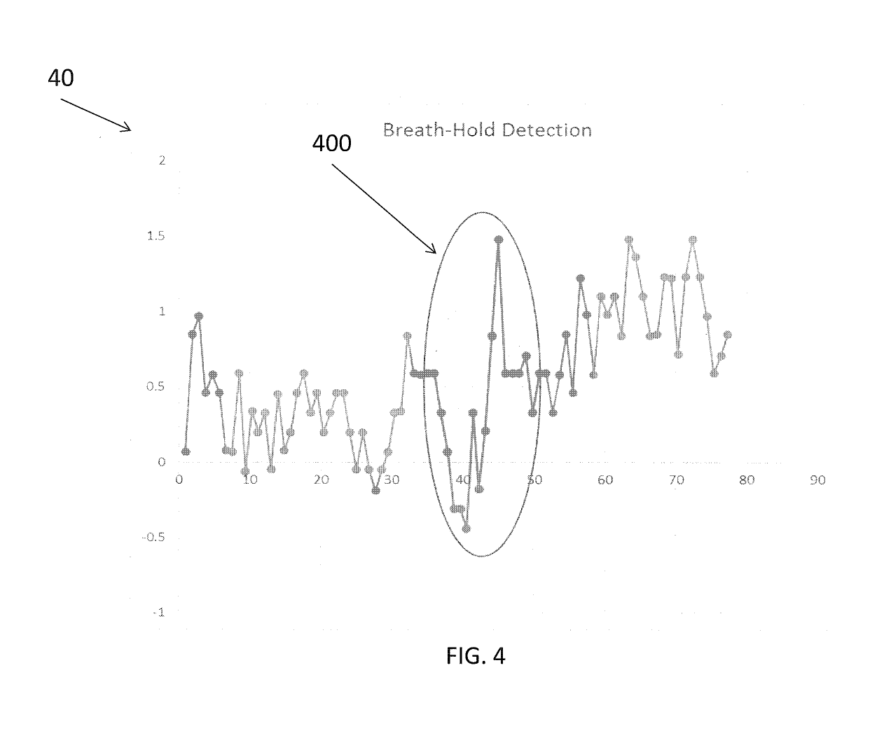

Furthermore, with the pulse rate sensor 204, inter-beat intervals (IBI) can be detected. The timing and length of individual hypoxic events can be determined from detection of IBIs with the pulse rate sensor. For example, a plot of breath holding detection of a user is shown in the graph 40 of FIG. 4. The hypoxic event (either induced by a breath training regimen or detected while the user is asleep) starts at time zero on the graph 40. The end of the hypoxic event is indicated by the circled area 400, where the change in heart rate, as detected by the pulse rate sensor 204, is indicative of the hypoxic event ceasing.

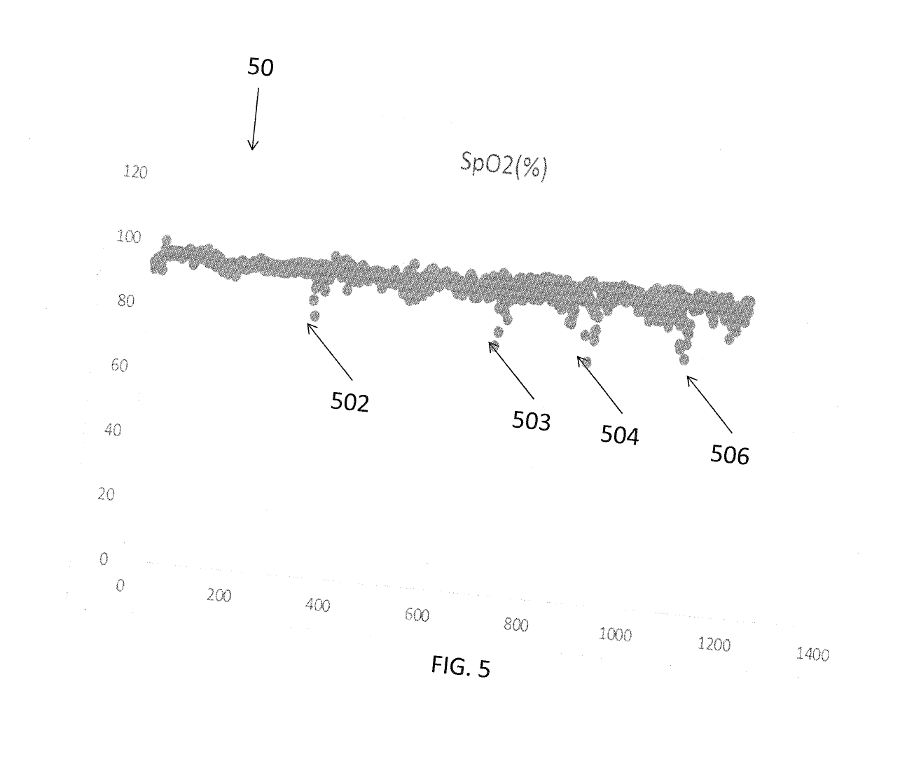

When an O2sat sensor is used, the data detected may be analyzed using a rolling average to seek reductions in the index from a baseline value. Periods of desaturation in the blood oxygen of a user can occur at both the start and end of a hypoxic event. This can be seen in FIG. 5. The chart 50 depicted in FIG. 5 show various desaturation events 502-506 in the user over many points in time over a time interval. Each desaturation event can be indicative of a hypoxic event, either occurring upon instruction of a breath training regimen or occurring while the user is asleep.

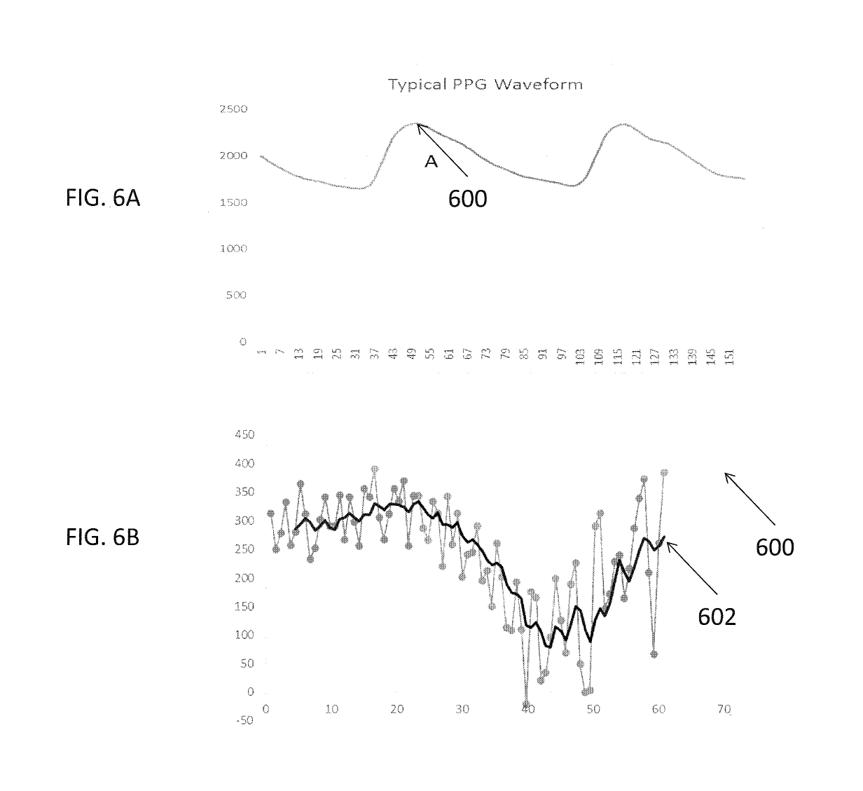

In embodiments where a PPG sensor is utilized, hypoxic events may be detected based on changes in amplitude of the PPG signal. FIG. 6A shows a typical peak 600 of a PPG waveform over two heart beats of the user. FIB. 6B is a chart 60 that shows changes in the PPG peaks over a time interval during a hypoxic event, and the change is depicted by the trend line 602. From the changes if PPG peak, the time, length, and occurrence of a hypoxic event can be detected, whether the event is voluntarily or involuntarily induced.

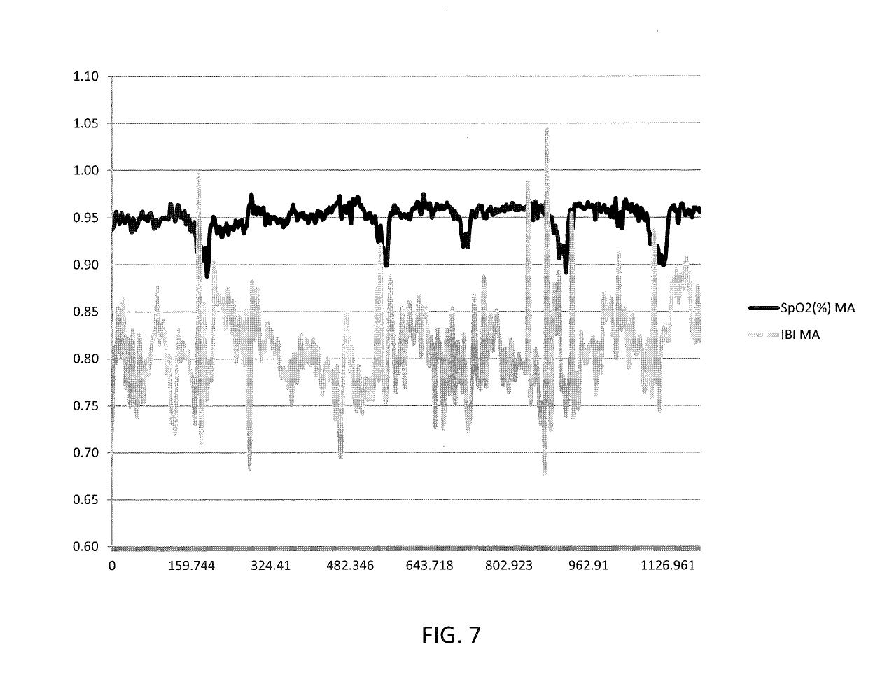

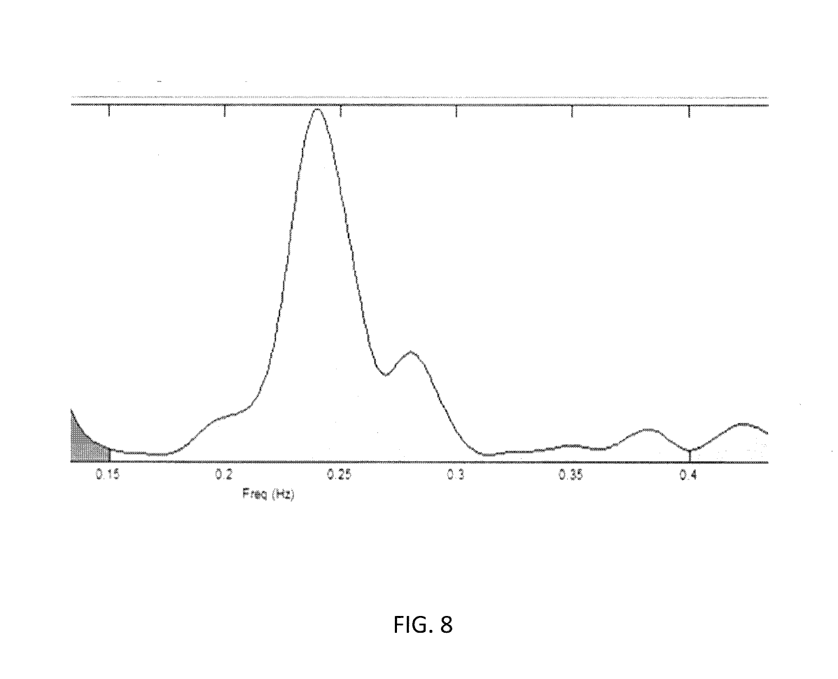

The characteristics of the physiological data detected by the sensor assembly 100, such as the PPG peaks, the O2Sat levels, the HRV, the IBI, etc., can be correlated together to not only determine individual hypoxic events over a time period, but determine trends in hypoxic events over the time periods based on changes in the characteristics over time. For example, FIG. 7 depicts both heart rate and O2Sat of a user after conversion from an asynchronous format to a synchronous format. This can be used to more closely determine the time of the hypoxic event. In examples where the hypoxic event is voluntarily induced via a breath training regimen, a more precise measurement of the breath holding time of the user can be determined by taking the difference between the time of the minima of the desaturation curve and the time that the user was prompted to exhale and hold their breath until they felt the need to breathe again.

The heart rate data in the form of interbeat intervals (IBI's) can be used to derive heart rate variability. In a healthy person, heart rate is known to vary synchronously with breathing rate. The greater this coherence, the more efficient is the pulmonary function and the more controlled is the autonomous breathing system. This is sometimes referred to vagal tone. To derive useful analysis of heart rate variability a Fast Fourier Transfer (FFT) is performed on the IBI data and subsequently filtered to derive a smoothed curve. One filter that is useful is the Welch filter. FIG. 8 shows such a FFT with Welch filtering for a patient with good coherence when breathing in a relaxed mode, such as may be found at the start of a typical breath training regimen or relaxed evenly paced breathing while asleep.

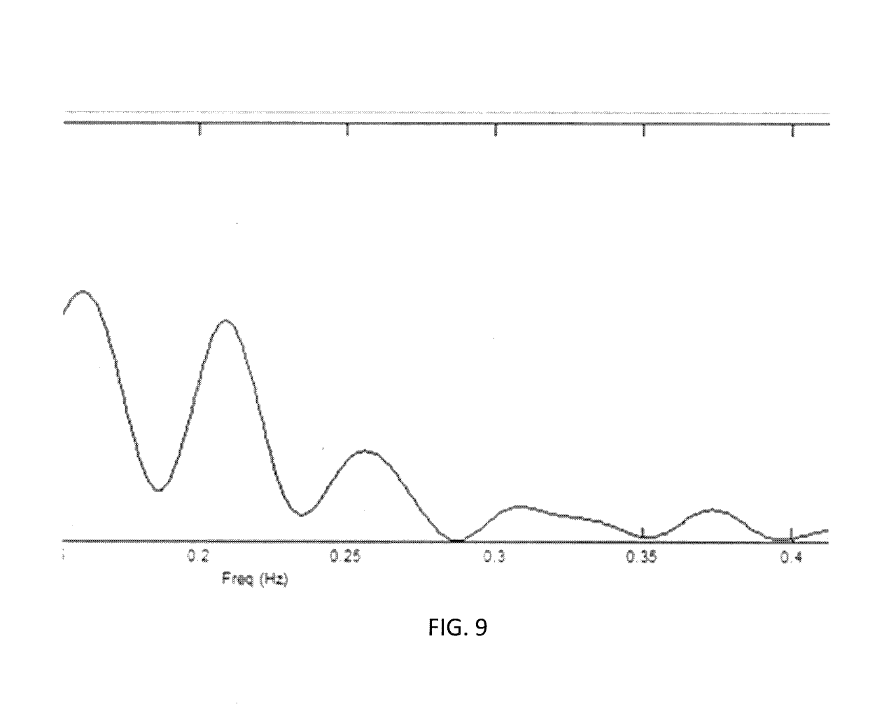

The same analysis methods can be used to determine the lack of coherence. This may occur during mild periodic hypoxic therapy or during other disturbed breathing episodes, such as sleep apnea, asthma and hyperventilation syndrome. FIG. 9 shows a FFT Welch filtered plot from a patient undertaking mild hypoxic therapy during imposed hypoxic events.

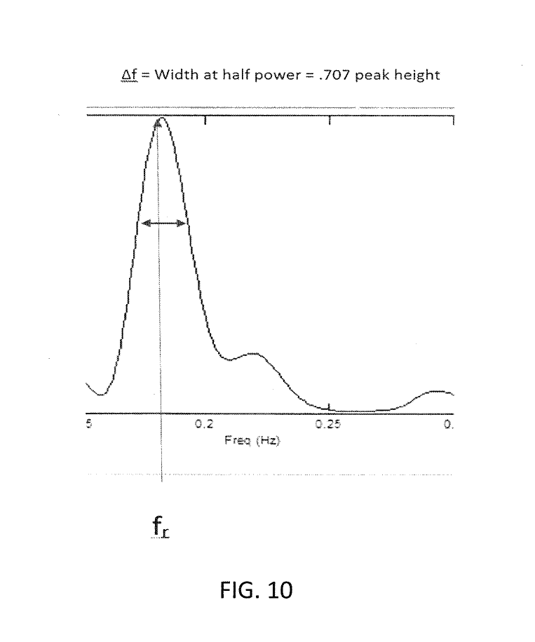

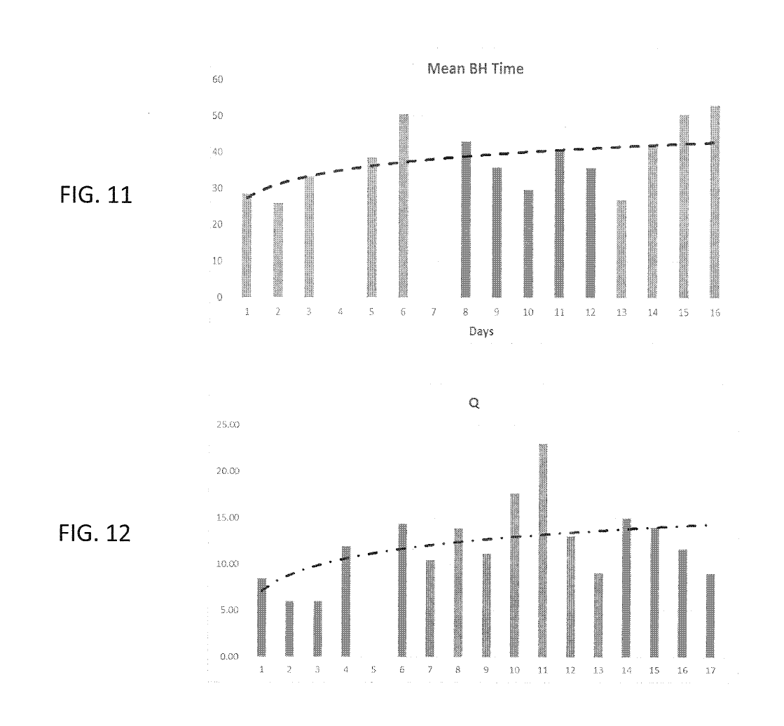

The strength of coherent breathing can be tracked by extracting the so-called Q value from the FFT filtered values of the HRV in the breathing frequency range. The Q factor is calculated by dividing the width of the resonance curve at half power by the frequency at the peak. Monitoring trends in Q can indicate changes in health, breathing efficiency, and the ability of the autonomous breathing control mechanisms to deal with challenges such as asthma attacks and sleep apnea events. FIG. 10 shows the vectors for calculating Q, in this case with a healthy patient with a high coherence factor.

The single regimen data described above are used to monitor trends during extended mild periodic hypoxia therapy. For example, FIG. 11 shows the mean daily breath hold data trending over time. A logarithmic trend curve is shown. Typically trend data during these therapies show such a trend form moving toward a final end value. Improved breath hold time indicates improvement in breathing control.

In one embodiment of the invention, self-imposed hypoxic events are monitored during breath training in the awakened state. The length of a hypoxic event caused by breath holding may be determined by sensing only the heart rate. Advantageously, hypoxia events can be monitored in the awakened state during breath training regimens while the user is not producing snoring events or periods of restlessness. In accordance with this embodiment, changes in heart rate occur during a period of self-imposed hypoxia. These changes can be in inter-beat interval changes and/or in the amplitude of peripherally sensed blood flow. The heart rate may be sensed using in-built sensors or cameras, e.g., such as those found in conventional smartphones such as the Apple iPhone available from Apple, Inc. of Cupertino, Calif. or the Samsung S6 phone available from Samsung Electronics America of Ridgefield Park, N.J.

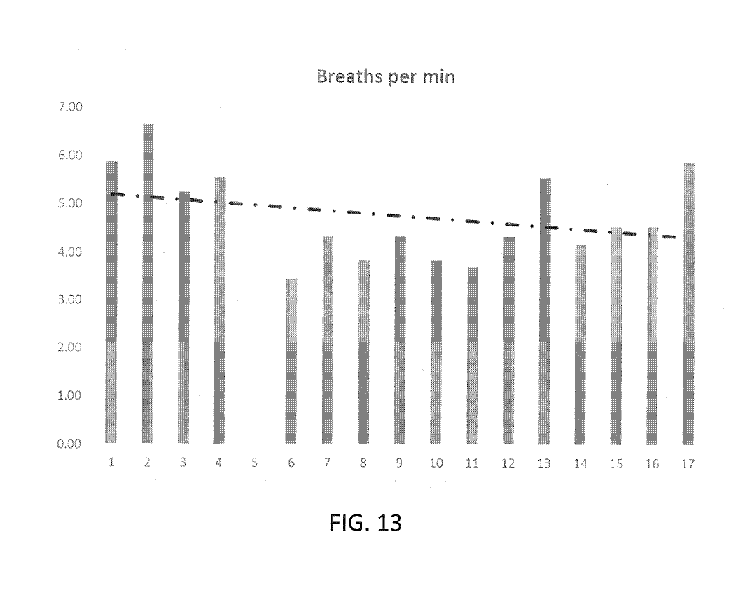

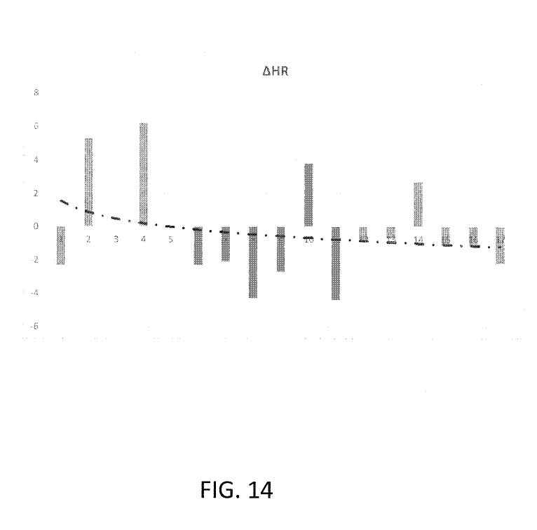

FIG. 12 shows the Change in Q value over time. Increase in Q indicates improved vagal tone, pulmonary health and breathing control. FIG. 13 shows changes in breaths per minute during relaxed breathing values over time. Decline in rest breathing rate indicates improved relaxation and breathing efficiency. FIG. 14 further shows changes in the heart rate decline that occurs between the start and finish of a single therapeutic session. A decline in heart rate during periodic hypoxia therapy indicates improved breathing control during breathing challenges.

Monitoring of SpO2 and heart rate during a therapeutic session and comparing the values with pre-determined limits can be used to prompt the patient to modify or terminate the session should these parameters exceed the limits. For example a heart rate exceeding 100 pulses per minute or a desaturation level below 90% of full saturation may be set as limits. Limits may be different for individual patients or at different times during the therapeutic process.

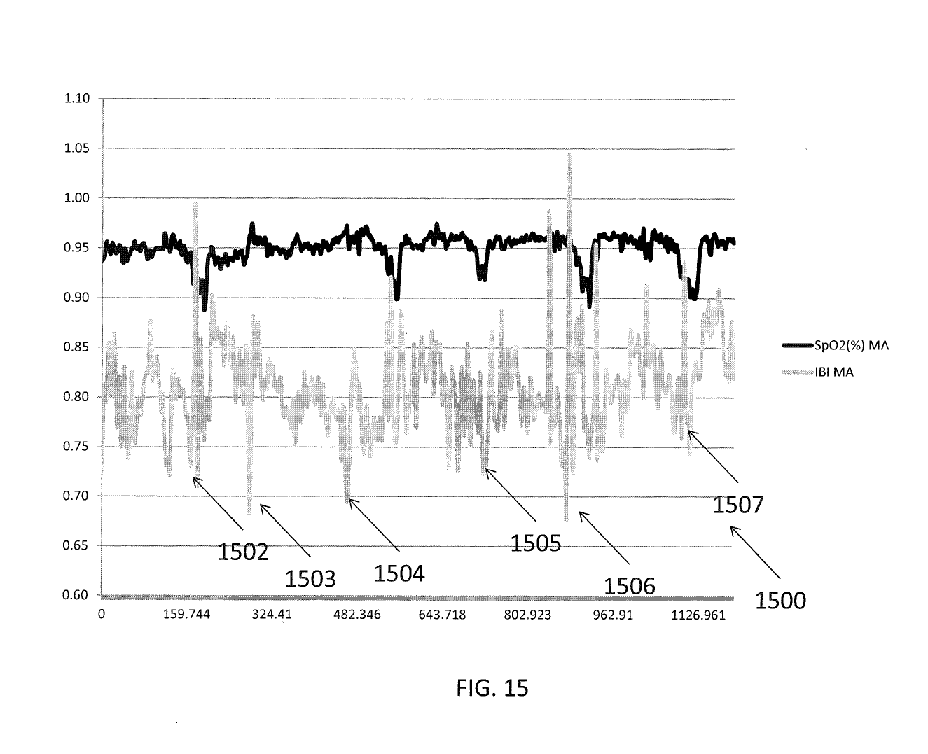

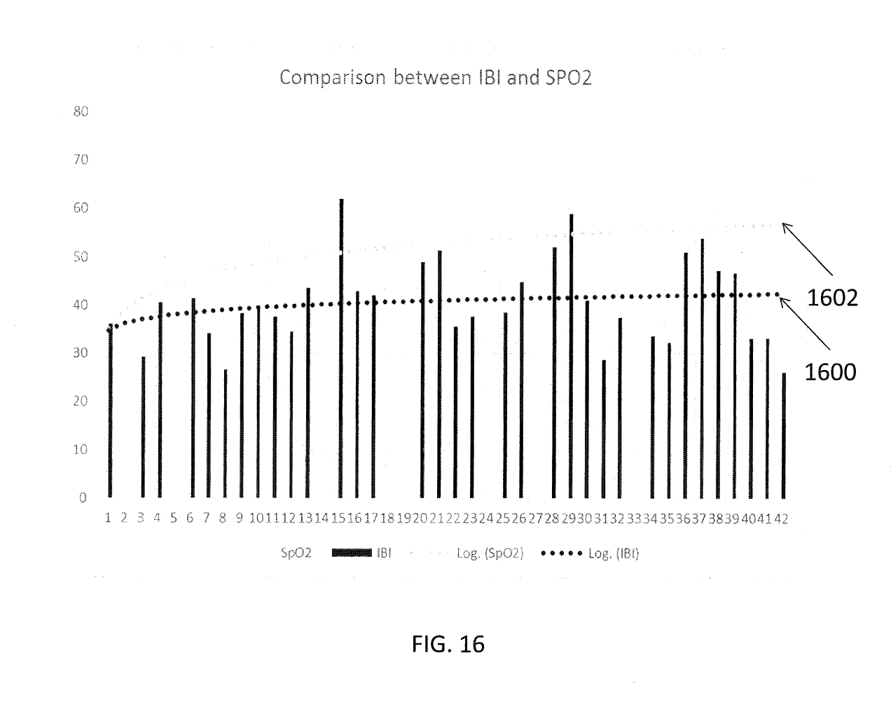

FIG. 15 shows plot 1500 of processed data from both IBI (exhibiting the higher excursions) and SpO2. The correlations between the two independent parameters can be seen at each of the six voluntarily imposed hypoxic events 1502-1507. FIG. 16 shows the lengths of a number of hypoxic events taken on subsequent days over a period of several weeks. The lengths gradually increase as shown by the trend curves 1600 and 1602. As the lengths increase, the difference between the IBI-based measurements and the SpO2-based measurements increase too. This is because the IBI values recover more rapidly than the SpO2 values after breathing recommencement, the latter requiring resaturation of blood oxygen at a peripheral location which is delayed from heart-rate recovery.

Along with each data point detected from the sensors of the sensory assembly 100, the system 10 may also include an electronic clock to associate times along with the physiological data detected by the sensor assembly 100. Thus, time signatures can be associated with each data point, which allows for better correlation between data detected from multiple sensors in the sensor assembly 100. In one example, data from an audio sensor (e.g., audio sensor 200) is analyzed using wavelet bi-coherence methods to determine whether periods of snoring are more likely to result from obstructive sleep apnea rather than being benign. The intensity and time span of the snoring events together with their time stamp of occurrence are used for subsequent correlation.

Advantageously, the systems, methods, and apparatus disclosed herein allow for correlation between multiple types of physiological data detected to increase the reliability of the data, reduce the appearance of errors in the data, and more effectively diagnose sleep disordered breathing by determining whether a sleep disordered event is benign or is indicative of a sleep disordered symptom.

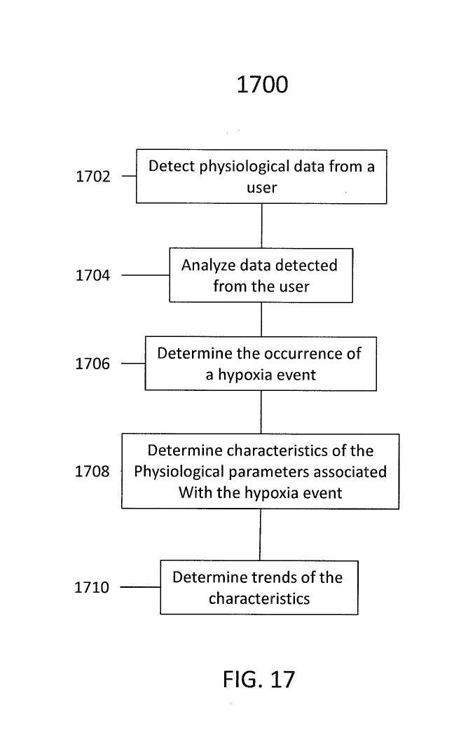

FIG. 17 is a flowchart 1700 of steps for monitoring hypoxia events of a user. At step 1702, physiological data is detected from the user. The data indicative of the physiological parameter may be detected from a sensor assembly.

At step 1704, the data detected is analyzed. The data is analyzed to remove factors such as outliers, inconsistencies, and other data infractions that may be present. Furthermore, the data indicative of one physiological parameter may be correlated with data of another physiological parameter to improve the accuracy of the analysis.

At step 1706, the occurrence of a hypoxia event is determined. The determination may be made based on the analysis performed at step 1704. The hypoxic event may be determined from data of one physiological parameter, or from correlation of data from multiple physiological parameters. As described above, particular characteristics of the physiological parameters may be indicative of hypoxic events.

At step 1708, characteristics of the physiological parameters used to determine the occurrence of a hypoxic event are determined. These characteristics are such as those described above. For example, changes in PPG amplitude, breath hold times, IBI, desaturation periods, etc. can be characteristics of the physiological parameters. These characteristics may be associated with the occurrence of the hypoxic event.

At step 1710, trends of the characteristics are determined. The trends are determined over a period of time or a time interval to establish whether the characteristics of the physiological parameters associated with the determined hypoxic events are improving or deteriorating over time. Based on the determined trends, updated or alternative breath training instructions/regimens may be provided to the user.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A system for monitoring hypoxia in a user over a period of time, the system comprising:
a sensor assembly configured to detect data indicative of at least one physiological parameter of the user,
an electronic computing unit comprising:
a processor configured to:
analyze the data indicative of the at least one physiological parameter of the user detected by the sensor assembly, and
determine, from the analyzed data, an occurrence of a hypoxia event;
determine characteristics of the data indicative of the at least one physiological parameter associated with the determined occurrence of the hypoxia event; and
determine trends from changes in the determined characteristics.

2. The system of claim 1, wherein the sensor assembly comprises at least one of a PPG sensor, a pulse rate sensor, and a blood oxygen saturation sensor.

3. The system of claim 1, wherein the processor is configured to determine trends by developing trend lines of the characteristics.

4. The system of claim 1, wherein the processor is configured to determine the occurrence of a hypoxia event by correlating data indicative of multiple physiological parameters.

5. The system of claim 4, wherein the multiple physiological parameters include blood oxygen level, breath hold time of the user, or pulse rate.

6. The system of claim 1, wherein the characteristics include at least one of PPG amplitude, blood oxygen desaturation, change in IBI, and breath hold time of the user.

7. A method for monitoring hypoxia in a user over a period of time, the method comprising:
detecting data indicative of at least one physiological parameter of the user;
analyzing the detected data indicative of the at least one physiological parameter of the user detected by the sensor assembly;
determining, from the analyzed data, an occurrence of a hypoxia event;
determining characteristics of the data indicative of the at least one physiological parameter associated with the determined occurrence of the hypoxia event; and
determining trends from changes in the determined characteristics.

8. The method of claim 7, wherein the determining trends step comprises determining trends by developing trend lines of the characteristics.

9. The method of claim 7, wherein the determining the occurrence of a hypoxia event comprises correlating data indicative of multiple physiological parameters.
